(19) United States
(12) Patent Application Publication
(10) Pub. No.: US 2026/0165609 A1
(43) Pub. Date: Jun. 18, 2026

(54) AUDITORY DISCRIMINATION SYSTEM

(71) Applicant: Maria L. K. Abramson, Laguna Niguel, CA (US)

(72) Inventor: Maria L. K. Abramson, Laguna Niguel, CA (US)

(21) Appl. No.: 19/416,754

(22) Filed: Dec. 11, 2025

Related U.S. Application Data
(60) Provisional application No. 63/734,035, filed on Dec. 14, 2024.

(51) Int. Cl. A61B 5/12; A61B 5/00

(52) U.S. Cl. CPC A61B 5/123; A61B 5/4836; A61B 5/7435; A61B 2503/06

(57) ABSTRACT

An auditory discrimination (AD) system is provided. The AD system is administered to a patient or subject through a series of listening trials that identify weakness or deficits in auditory discrimination of the patient. The AD system also provides targeted training trials for remedying the auditory deficit and improving auditory discrimination. Since the patient or subject of the AD system is often a young child, the AD system verifies that the subject has the concept of “same” versus “different” and can indicate when two sounds are the same versus when two audio tones are different. Upon successfully verifying that the subject can indicate whether two sounds are the same or different, a first set of N trials is administered to the subject as training for a pitch discrimination test (PDT), and then a second set of M trials is administered to the subject as the PDT.

### CROSS REFERENCE TO RELATED PATENT APPLICATION(S)

The present disclosure is part of a non-provisional application that claims the priority benefit of U.S. Provisional Patent Application No. 63/734,035, filed on Dec. 14, 2024, titled “Auditory Discernment System”.

### BACKGROUND

### Technical Field

The present disclosure generally relates to a hearing diagnostic and treatment system, specifically based on auditory discrimination or pitch discrimination.

### Description of Related Art

Research in auditory development demonstrates that frequency discrimination is present during fetal stages and continues to mature throughout infancy and childhood. Fetuses at approximately thirty-five weeks of gestational age can differentiate tonal frequencies such as 250 Hz and 500 Hz, and infants born between thirty and thirty-five weeks exhibit cortical mismatch-negativity responses to changes in speech sounds. The frequency difference limen for a 1000 Hz tone typically ranges from 11 to 19 Hz in infants and from 3 to 13 Hz in adults, indicating progressive refinement of auditory discrimination with maturation. Auditory system development proceeds from peripheral to central neural pathways and encompasses successive stages of sound coding, selective listening, and perceptual flexibility. During early language acquisition, infants perceive speech across a broad spectral range and subsequently focus on native-language phonetic prototypes by approximately six months of age. These findings establish frequency discrimination as a core auditory function essential to phonological development, speech perception, and higher-order language processing.

Studies of auditory maturation further demonstrate that discrimination of sound intensity develops later than frequency discrimination and relies on progressively higher levels of neural processing. It is also observed that discrimination of pitch is more robustly represented and encoded within auditory neural pathways than discrimination of sound intensity, reflecting the greater developmental and functional significance of pitch-based auditory processing for speech and language acquisition.

Pitch discrimination represents a critical component of auditory development and forms the perceptual foundation for language, reading, and other complex auditory skills. Psychoacoustic studies demonstrate that, following basic sound detection, pitch discrimination is among the most essential auditory abilities and is integral to speech and phonological processing. Deficits in pitch discrimination have been associated with impairments in phonological awareness, dyslexia, auditory memory, and various language and learning disorders. The capacity to discriminate pitch contributes directly to the development of phonemic recognition, spelling, and reading proficiency by enabling accurate perception of temporal and spectral features of speech. Because of its fundamental role in auditory perception and its measurable relationship to developmental outcomes, evaluation of pitch discrimination should be included within comprehensive assessments of central auditory processing, particularly in children at risk for auditory, reading or language-related deficits.

Discriminating pitch, loudness, and timing is essential for normal speech and language development, phonological processing, and reading acquisition. Temporal resolution is a critical aspect of auditory processing, as the ability to perceive brief acoustic events supports accurate identification of phonemes and syllabic patterns. Research in auditory science has shown that the fastest English vowels are approximately 100 milliseconds in duration, and that a temporal gap of about 300 milliseconds between successive tones closely aligns with the auditory temporal processing requirements necessary for speech perception. These temporal parameters define the interval within which the auditory system integrates sequential acoustic information and therefore serve as physiologically and linguistically relevant benchmarks for evaluating and training auditory discrimination performance.

### SUMMARY

Building upon these established temporal spectral cues and perceptual foundations, some embodiments of the disclosure provide an auditory discrimination (AD) system that is administered to a patient or subject through a series of listening trials that identify weakness or deficits in auditory discrimination of the patient. The AD system also provides targeted training trials for remedying the auditory deficit and improving auditory discrimination. The AD system is designed to test the patient's ability to discriminate sound based on pitch and timing presented to the patient at a comfortable listening volume. In some embodiments, the AD system includes a testing protocol that is set up to identify frequencies or frequency ranges that the patient has difficulty discriminating differences in pitch. Such a testing protocol may be referred to as a pitch discrimination test (PDT).

Since the patient or subject of the AD system is often a young child, the AD system verifies that the subject understands the concept of “same” versus “different” and can indicate when two sounds are of the same frequency versus when two audio tones are of different frequencies. Upon successfully verifying that the subject can indicate whether two sounds are the same or different, a first set of N trials is administered to the subject, each trial having two audio tones. Once the subject is verified to have correctly indicated which of the first set of N trials have two same-frequency audio tones and which of the N trials have two different-frequency audio tones, the system provides a second set of M trials to the subject, each trial having two audio tones (M is at least twice large as N, e.g., N≤4 and M≥10). In some embodiments, the first set of N trials are used as training to take the PDT (the PDT training), while the second set of M trials are used as the pitch discrimination test (the PDT test).

After administering the second set of M trials (the PDT test) to the subject, the system receives an assessment based on the subject correctly indicating which of the second set of M trial(s) have/has two same-frequency tones and which trial(s) have/has two different-frequency tones. An audio discrimination training program may then be prescribed for the subject based on the assessment, which may be part of the treatment for a hearing issue of the subject. In some embodiments, the system may perform the audio discrimination training program with the subject based on the assessment.

### BRIEF DESCRIPTION OF THE DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 conceptually illustrates an auditory discrimination (AD) system providing pitch discrimination test (PDT) to a patient or subject.

FIG. 2 conceptually illustrates a process for implementing PDT by an AD system.

FIG. 3 illustrates an example script that can be followed by a human operator as he or she works with a patient through the PDT training.

FIG. 4 illustrates an example form in which the results of the PDT training trials can be recorded.

FIG. 5 illustrates an example form in which the result of PDT test trials can be recorded.

FIG. 6 provides a listing of the supplemental tracks used by the AD system in some embodiments.

FIG. 7 shows an example aged-based percentile mapping from PDT test scores.

FIG. 8 conceptually illustrates an electronic system with which some embodiments of the present disclosure are implemented.

### DETAILED DESCRIPTION

Psychoacoustic literature shows that, next to detection, frequency discrimination is the most important psychoacoustic ability, as it is necessary for speaking, pre-reading, reading and spelling skills. It is recognized that assessment of auditory discrimination be considered as part of a test battery for (central) auditory processing disorders. Auditory discrimination (AD) plays a fundamental role in the development of speaking, pre-reading, reading, language and more complex auditory processes. Frequency discrimination is important for basic sensory processing, affecting phonological processing, dyslexia, measurements of intelligence, auditory memory, Asperger's Syndrome, and other language impairments. AD is such a basic auditory process that it factors into virtually every test of central auditory function, deficits with auditory discrimination can result in language, reading and spelling problems.

AD may be defined by a variety of behavioral and objective measures which may include the following: same/different judgment tasks, tasks identifying just noticeable differences between stimuli (DL or JND) and time order judgments (TOJ) of which sound came first. Auditory discrimination can be assessed in terms of discriminating frequency, intensity or timing differences.

Auditory discrimination of frequency is very important to children learning to extract the phonetic code from spoken language based upon their ability to process temporospectral cues, while poor phonological processing underlies language and/or reading skills deficits. Processing frequency cues combined with temporal processing, i.e., temporal spectral cues, are the building blocks of phonological development. The brain's ability to process each phoneme's characteristic acoustic temporospectral feature occurs during the critical period of speech and language acquisition. It is reported that early auditory deficits, differences or experiences are likely to affect the sharpness of phonological representations that are established through experience-dependent learning in infancy, leaving a lasting effect of phonological impairment. Frequency discrimination appears developmental in nature and is related to intelligence, reading, auditory memory, and certain specific language impairment. Frequency discrimination has a widespread influence upon the development of speaking, pre-reading skills, reading and spelling skills. Thus, there is a critical need for tests of auditory discrimination, specifically frequency discrimination for young children, in an audiologist's test battery.

Some embodiments of the disclosure provide an auditory discrimination (AD) system that is administered to a patient or subject through a series of listening trials that identify weaknesses or deficits in auditory discrimination of the patient. The AD system also provides targeted training trials for remedying the auditory deficit and improving auditory discrimination. The AD system is designed to test the patient's ability to discriminate sound based on pitch and, with all tones presented at the patient's most comfortable listening level. In some embodiments, the AD system includes a testing protocol that is configured to identify frequencies or frequency ranges that the patient has difficulty discriminating. Such a testing protocol may be referred to as a pitch discrimination test (PDT).

Since the patient or subject of the AD system is often a young child, the AD system verifies that the subject understands the concept of “same” vs “different” by verifying that the subject can identify when two similar objects are of the same color vs different colors. After the subject demonstrates the understanding of “same” vs “different” the subject is questioned if he/she can indicate when two sounds produced using a visual cue (e.g. xylophone with colored tone bars) are the same pitch versus when two audio tones with a visual cue are of a different pitch. After the subject has practiced answering whether two sounds are the same or different, a first set of N trials of the PDT training is administered to the subject, each trial having two audio tones. Once the subject has practiced indicating which of the first set of N trials have two same-frequency audio tones and which of the N trials have two different-frequency audio tones, the system provides a second set of M trials to the subject, each trial having two audio tones (M is at least twice large as N, e.g., N≤4 and M≥10) and the results of the M trials recorded as correct or incorrect. In some embodiments, the first set of N trials are used as training to take the PDT (the PDT training), while the second set of M trials are used as the pitch discrimination test (the PDT test).

After administering the second set of M trials (the PDT test) to the subject, the system receives an assessment based on the subject correctly indicating which of the second set of M trial(s) have/has two same-frequency tones and which trial(s) have/has two different-frequency tones. An audio discrimination training program may then be prescribed for the subject based on the assessment, which may be part of the treatment for a hearing issue of the subject. In some embodiments, the system may perform the audio discrimination training program with the subject based on the assessment.

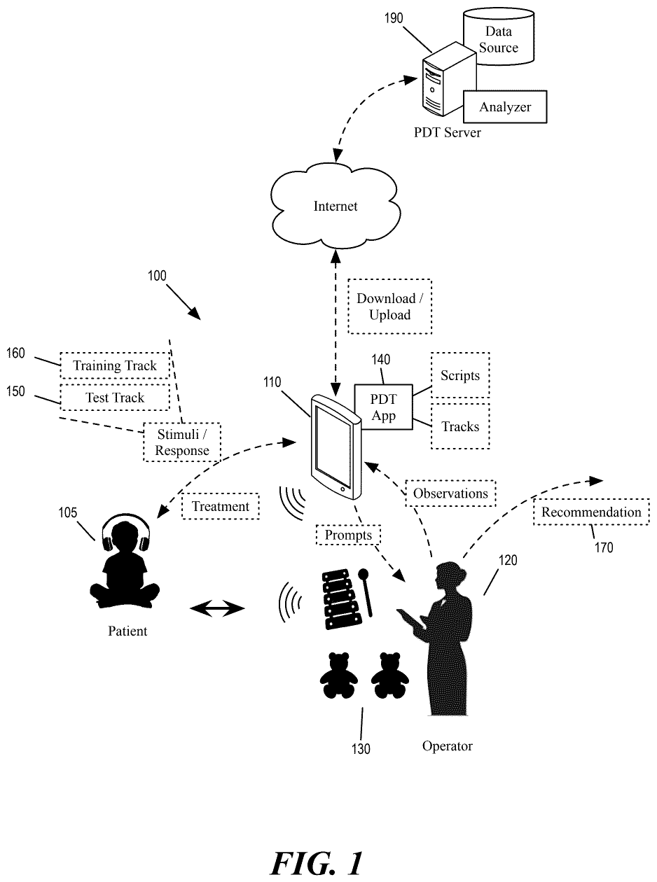

In some embodiments, the AD system providing the PDT test is implemented by a computing device that is operated by a human operator. FIG. 1 conceptually illustrates an AD system 100 providing PDT test to a patient or subject 105, who may be a child in an age range of age 3 years or older. As illustrated, the AD system 100 is implemented by a computing device 110. The computing device 110 presents various auditory and visual stimuli to the patient 105 at its output devices (e.g., display screen, audio speakers, earphone, etc.) The computing device 110 is operated by a human operator 120, who interacts with the patient 105 as guided by prompts or scripts provided by the computing device 110. The human operator 120 may also employ physical items 130 to provide some of the auditory and visual stimuli (e.g., xylophones, teddy bears). Through his or her interaction with the patient 105, the human operator 120 may report the patient's response to the AD system 100, along with his or her observations of the patients. In some embodiments, the patient may also directly interact with the computing device 110 (by e.g., touch screen, keyboard, etc.) to provide the response. The AD system 100 in turn uses the reported response and observations to provide diagnostic trials, result analysis, treatment prescription, treatment programs, and other treatment recommendations. These reports and recommendations 170 can be generated by the computing device 110 or made by the human operator.

The computing device 110 may be a mobile device, a tablet, a desktop, a laptop, a workstation, or any other types of computing device. The computing device 110 is connected to the Internet and may download software applications from through the Internet. To implement the AD system 100, the computing device 110 may download and run a PDT app 140. The PDT app 140 includes various audio and video tracks/clips to be presented to the patient 105 as audio and visual stimuli. Such stimuli may include test tracks 150 and training tracks 160, which will be further described in Section C below.

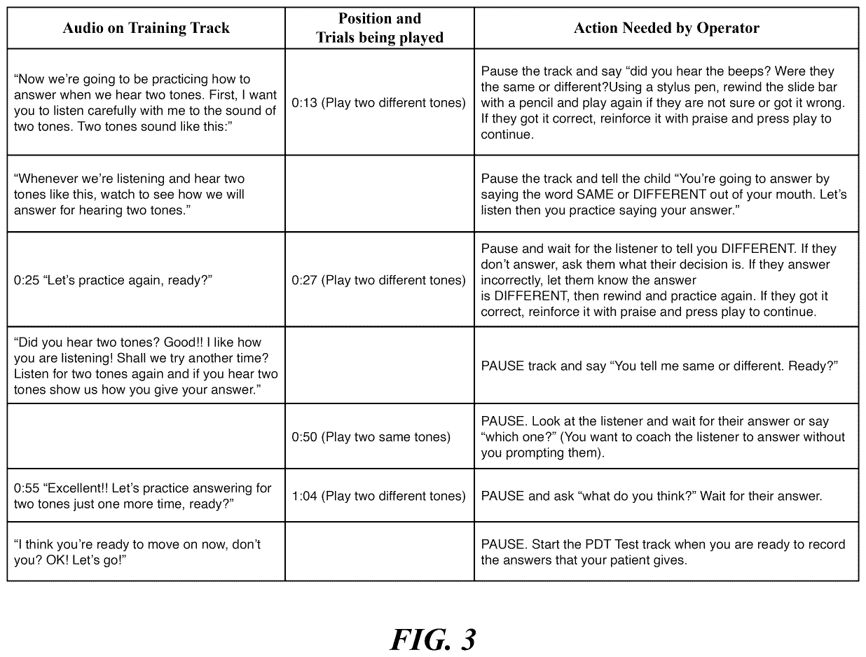

In some embodiments, the PDT app 140 may also include scripts that are presented as prompts to guide the human operator 120. An example of such a script is shown in FIG. 3 below. In some embodiments, the responses and the observations may be uploaded to a PDT server 190 to perform more complex analysis, including by artificial intelligence systems. In some of these embodiments, the PDT server 190 is also the data source for providing up-to-date versions of the PDT app 140 with updated data (e.g., stimuli and scripts) to be downloaded by the computing device 110.

In some embodiments, the various tracks of the AD system (training, test, therapy, supplemental) are stored in a computer readable storage medium, which may include, but is not limited to, RAM, ROM, EEPROM, flash memory or other memory technology, CD-ROM, digital optical disks or other optical storage, magnetic cassettes, magnetic tape, magnetic disk storage or other magnetic storage devices, or any other non-transmission medium that can be used to store information for access by a computing device. In some embodiments, the AD system stores at least some of the audio tracks in a server for download into a computing device for later playback.

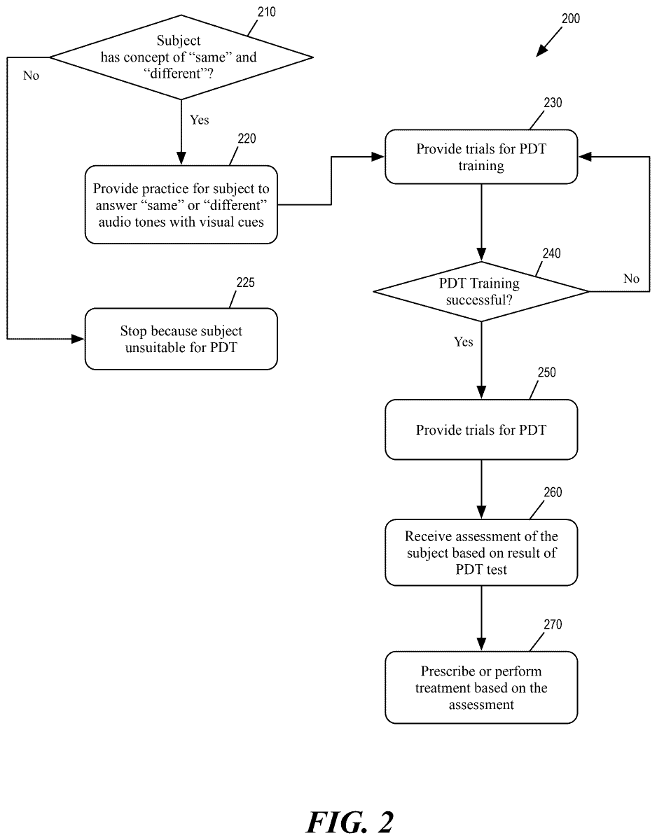

FIG. 2 conceptually illustrates a process 200 for implementing PDT by an AD system. In some embodiments, one or more processing units (e.g., a processor) of a computing device implementing the AD system 100 performs the process 200 by executing instructions stored in a computer readable medium. In some embodiments, an electronic apparatus implementing the AD system performs the process 200. In some embodiments, a human operator using the AD system (e.g., the human operator 120) may perform some or all steps of the process 200.

The system determines (at block 210) whether the subject has the concept of “same” and “different”, specifically by verifying the subject can indicate when two objects are visually the same versus when two objects are visually different. The child may be asked to indicate whether two items are same or different by the human operator. The two items may be physical items (such as toys), virtual items displayed by a computing device or a picture. The subject's response may be reported to the system by the human operator or captured by the computing device. The system may verify whether the subject has the concept of “same” or “different” based on the subject's response directly or based on the human operator's reported observation and assessment. The operations described by reference to block 210 are further described in Section A (Fundamental Skill Verification) below. If the subject is verified to have the concept of “same” and “different”, the process proceeds to block 220. If the subject does not appear to have the concept of “same” and “different”, the process proceeds to block 225 to stop PDT because it is not suitable for the subject.

The system provides (at block) practice for the subject to answer “same” or “different” when presented with audio tones with a visual cue. Specifically, the subject is asked to indicate when two audio tones are the same versus when two audio tones are different, with each tone generated and presented to the subject in conjunction with a visual cue (e.g. by striking colored tone bars on a xylophone). The child may be asked to indicate whether the two audio tones are same or different by the human operator. The two audio tones can be generated by a physical musical instrument such as a xylophone or be generated by the computing device which presents a visual cue. The subject's response may be reported to the system by the human operator or captured by the computing device. The system may verify whether the subject understand “same” and “different” audio tones with a visual cue based on the subject's response directly or based on the human operator's reported observation and assessment. Operations described by reference to blocks 220 are further described in Section A (Fundamental Skill Verification) below. The process then proceeds to block 230.

The system provides (at block 230) trials for PDT training (with coaching) for the purpose of conditioning the subject for the PDT test. In some embodiments, the system provides a set of N trials (e.g., N≤4), each trial comprising two audio tones that may have the same frequency or different frequency. The trials may be administered to the subject by the human operator controlling the computing device. The human operator may interact with the subject according to a script provided by the AD system and make observations of the subject. PDT training is further described in Section B below. In some embodiments, the trials for the PDT training are packaged as audio tracks that are referred to as training tracks. Training tracks are further described in Section C below.

The system determines (at block 240) whether the PDT training is successful by e.g., verifying that the subject has correctly indicated which of the set of N trials have two same-frequency audio tones and which of the N trials have two different-frequency audio tones. In some embodiments, PDT training is considered successful all the subject correctly indicated all N trials. The system may verify whether PDT training is successful based on the subject's response directly, or based on the subject's score reported by human operator. If the PDT training is successful, the process proceeds to block 250. If the PDT training is unsuccessful, the process may return to block 230 to perform PDT training with the subject again by repeating the PDT training trial. In some embodiments, regardless of whether the PDT training is successful or unsuccessful, after the second attempt of the PDT training trial (or after a specific number of attempts), the process proceeds to block 250.

The system provides (at block 250) trials for PDT test, specifically by providing a second set of M trials (e.g., M≥10; M is least twice large as N in some embodiments), each trial having two audio tones that may have the same frequency or different frequency. The trials may be administered to the subject by the human operator controlling the computing device. The human operator may interact with the subject according to a script provided by the AD system and make observations of the subject. The PDT trials may be conducted without coaching. The subject's response to the PDT trials are recorded as the result of the PDT test. PDT test is further described in Section B below. In some embodiments, the trials for PDT test are packaged as audio tracks that are referred to as test tracks. Test tracks are further described in Section C below.

The system receives (at block 260) an assessment of the subject based on the result of the PDT test, specifically based on the subject correctly indicating which of the second set of M trials have two same-frequency tones and which trial have two different-frequency tones. In some embodiments, the assessment utilizes a percentile score that is determined on the subject's age. In some embodiments, the human operator makes the assessment based on the subject score of the PDT test and reports the assessment to the system. In some embodiments, the system tabulates the score of the PDT test based on the subject response and makes the assessment. Such an assessment may be further based on observations reported by the human operator.

In some embodiments, the system may utilize an artificial intelligence system at a remote server (e.g., the PDT server 190) to generate the assessment based on the PDT test score and the observations. Assessment of the subject based on PDT test is further described in Section D below.

The system prescribes (at block 270) or perform treatment for the patient based on the assessment. For example, the system may be used to monitor the subject by periodically performing a pitch discrimination test with the subject, namely providing trials with each trial comprising two audio tones, and receiving an assessment based on the subject correctly indicating which of the trials have two same-frequency tones and which of the plurality of trials have two different-frequency tones. In some embodiments, the system performs audio discrimination training with the subject based on the assessment, for example, by repeating trials that the subject had difficulty distinguishing “same” versus “different” and training the subject to distinguish tones in those frequency ranges. Section E below further describes treatment of the patient based on the PDT test assessment.

### A. Fundamental Skill Verification

In some embodiments, the AD system verifies that the patient has certain foundational skills for using the AD system and to condition the patient to be able to use the AD system. For some embodiments, this is a two-step process: (1) verifying whether the subject understands when two objects are visually the same versus when two objects are visually different, and (2) verifying that the subject can indicate when two sounds are the same versus when two sounds are different.

The system ensures that the patient (who may be a young child) has the concept of “same” and “different”. The system also ensures that the patient can verbalize or otherwise communicate his or her judgement whether two things/objects (e.g., two sounds) are “same” or “different”. The system may use visual and auditory queues to prepare or prime the patient for indicating their judgement on whether two objects or sounds are “same” or “different”. In some embodiments, if the system determines that the patient does not have the concept of “same” and “different” and/or is not able to verbalize or communicate his or her judgement for sounds, the system may indicate to the user that the AD system is not suitable for the patient so the user may choose to terminate the system.

The system may provide a user interface for the patient to indicate “same” versus “different”, verbally, or by physically pointing at a graphical user interface (GUI) element. In some embodiments, a computing device implementing the AD system visually presents the two objects at a screen of the computing device. Thus, the system may verify that the patient (who can be a very young child) knows the concept of same and different by presenting teddy bears (e.g., as images on a display device) to the patient and then asks the patient to identify teddy bears having the same color and teddy bears having different colors. In some embodiments, a human operator may present two physical items (such as two physical teddy bears) to the subject and ask the subject to indicate whether the two physical items have the same or different colors. The subject's response may then be reported to the system (e.g., the computing device implementing the AD system) to determine whether to proceed (i.e., whether the AD system is suitable for the patient.) Such determination may also be made by the human operator administering the AD system.

Once the subject is verified to have the concept of “same” versus “different” and can indicate so, the system may verify whether the subject can indicate when two sounds (e.g., audio tones) are the same versus when the two sounds are different when presented with a visual cue. In some embodiments, a computing device implementing the AD system may audibly present (play) the two sounds (e.g., two audio tones) at a speaker of the computing device. In some embodiments, the human operator may use a physical instrument (such as a xylophone) to generate the two sounds or two audio tones with a visual cue. The human operator may ask the child whether the two sounds are the same or different and decides whether to proceed with the AD system. The human operator may enter the subject's response into the computing device implementing the AD system, which may indicate whether the subject is ready to proceed. The human operator may also allow the subject to indicate whether the two sounds are the same or different by letting him or her interacting with the GUI the computing device. For example, in some embodiments, the AD system may visually present a keyboard or a xylophone for generating sounds of different pitches.

More generally, the system verifies that the patient recognizes that sound has properties, and that are making a judgement based on the property of the sound. The subject listens to a sound and makes a perceptual judgement of that sound. The system then generates sounds or tones while showing which corresponding keys are pressed. This is to show the patient that sounds can have similar pitches (e.g., same key or neighboring keys) or dissimilar pitches (e.g., when keys are far apart). In some embodiments, the patient may be asked to discriminate sounds that are generated with some time interval apart and have a specific duration (e.g., 300 or 500 milliseconds). This requires that the patient hold the sound in his or her auditory memory as the patient makes the perceptual judgement.

### B. PDT Training and Test

In some embodiments, once the patient shows that he or she understands the concept of “same” versus “different” by identifying same/different color items and audio tones, PDT training is administered to condition or teach the patient to answer with either the word “same” or the word “different” after they hear two tones. The PDT training is conducted by using a training track provided by the AD system, which includes trials of pairs of audio tones as described below in Section C. Some patients prefer to answer “same” and “not the same”, or “yes” and “no”. When a patient demonstrates that his/her perceptual judgement is consistently accurate, these alternative ways of answering are acceptable. FIG. 3 illustrates an example script that can be followed by a human operator (e.g., a therapist or an audiologist) as he or she works with a patient through PDT training. The script will require the PDT training to start and stop several times. If the patient provides an incorrect answer, an operator may rewind the training track to replay the tones that the patient got wrong. In some embodiments, rewinding the track is accomplished by using a slide bar on an app running on the computing device implementing the AD system. In some embodiments, PDT training is considered completed by the AD system when all trials (e.g., 4 trials) of the PDT training are complete, i.e., the patient has successfully indicated which of the audio tone pairs are “same” and which of the audio tone pairs are “different”.

After the PDT training is completed, the actual PDT test may be administered by conducting a set of trials (e.g., 10 trials). For each trial, the AD system plays a pair (two beeps) of sounds or tones and then prompts the patient for a discrimination response, e.g., to indicate whether the two beeps in the pair are the same or different. After administering the PDT test, the AD system may identify which trials or which frequencies that the subject fails.

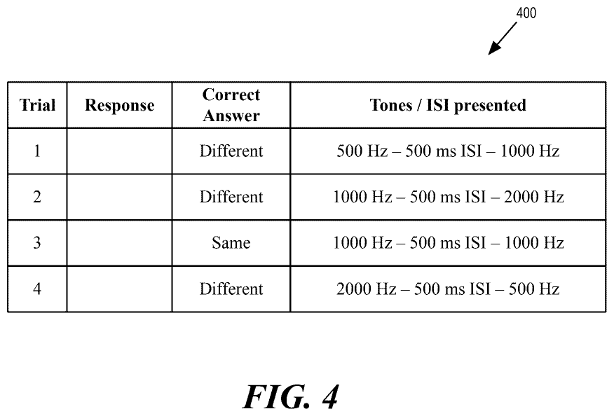

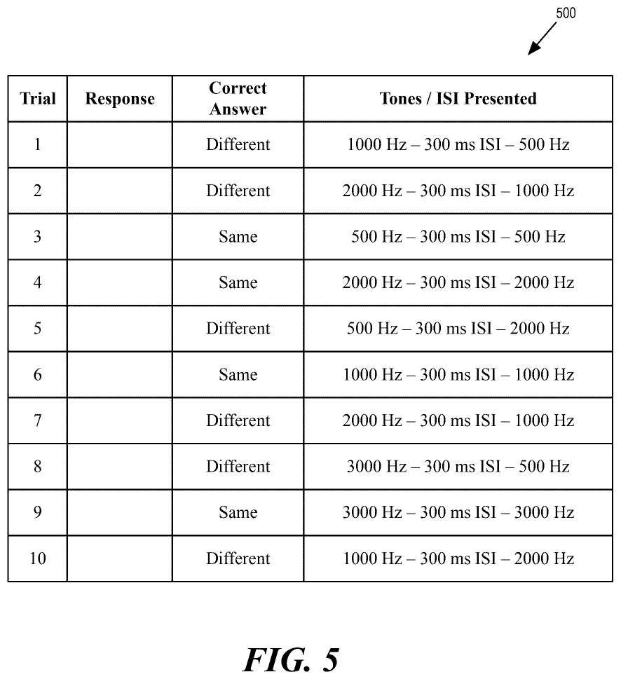

FIG. 4 illustrates an example form 400 in which the results of the PDT training trials can be recorded. FIG. 5 illustrates an example form 500 in which the result of PDT test trials can be recorded. Each row of the forms corresponds to a trial and shows the following information for the trial: (i) the response of the subject; (ii) the correct answer (“same” or “different”); (iii) the frequencies of the two audio tones (beeps) of the trial; and (iv) the duration of the ISI between the beeps.

### C. System Audio Tracks

In some embodiments, the AD system uses a multitude of system audio tracks to conduct the trials. Each system audio track includes several trials, each trial including a pair of beeps (or audio tones) that may be of the same frequency, or of different frequencies. The trials may have different combinations of sounds, e.g., a first trial may have a beep pair {1000 Hz, 2000 Hz}, and a second trial may have beep pair {1500 Hz, 1500 Hz}, etc.

In some embodiments, the system audio tracks use trials having audio tones in a frequency range between 500 Hertz and 3000 Hertz. In some embodiments, the system audio tracks include at least one trial having two audio tones that differ in frequency by more than 750 Hertz and less than 1250 Hertz. In some embodiments, the test and training tracks include at least one trial having two audio tones that differ in frequency by less than 750 Hertz but more than 250 Hertz. In some embodiments, between the two beeps of each pair is an ISI (inter stimulus interval) of a specified time duration (e.g., 300 milli seconds for the test trials and 500 milliseconds for the training trials).

The system audio tracks may include training tracks (for PDT training) and test tracks (for PDT test). The test tracks are used to test the current auditory discrimination ability of the patient. A test track includes a series of trials that is a mixture of trials with same frequency pairs and trials with different frequency pairs. The training tracks are used to condition or train the patient to answer with either the word “same” or the word “different” after they hear two tones. In other words, the training tracks is used to prepare the patient to take the PDT test. In some embodiments, the training tracks are generated by sub-sampling the test tracks, e.g., by taking some of the trials of the test tracks. In some embodiments, the AD system may generate randomized trials with “same” and “different” beep pairs to perform auditory discrimination testing or training. In some embodiments, the trials in a test track are at least twice as numerous as the trials in a training track. In some embodiments the inter stimulus interval is longer for the trials in the training track vs the trials in the test track (e.g., 500 ms on the training track vs 300 ms on the test track).

In some embodiments, the system audio tracks also include therapy tracks and supplemental tracks. The therapy tracks are used to improve the patient's auditory discrimination ability. The therapy tracks may include “same” tracks and “different” tracks for training the patient to recognize same and different sounds (e.g., in terms of pitches or frequencies). Each track of the “same” tracks and “different” tracks includes a series of trials. Each trial in a “same” track includes a pair of beeps that are of the same frequency, while each trial in a “different” track includes a pair of beeps that are of different frequencies. When playing a “same” track, the AD system may inform the patient that the he or she is listening to a series of beep pairs that are the same. The AD system may play the “different” tracks after informing the patient he or she is listening to a series of beep pairs that are different. In some embodiments the training tracks have both “same” and “different” pairs in a random sequence.

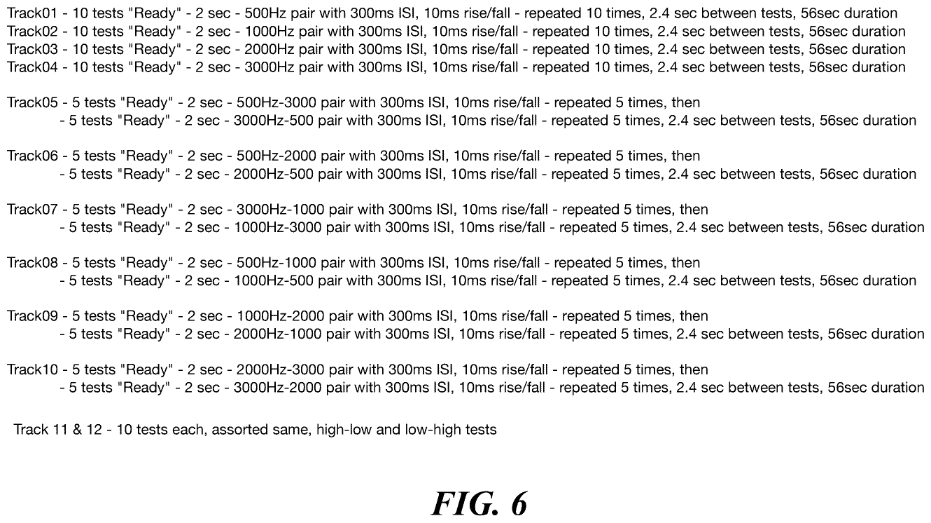

The supplemental tracks, also referred to as faster tracks, include “different” tracks and “same” tracks that have shorter time separating trials than training tracks. For example, in some embodiments, the trials in training tracks are separated by 6.8 seconds, while the trials in the supplemental trials are separated by 2.4 seconds. FIG. 6 provides a listing of the supplemental tracks used by the AD system in some embodiments.

### D. Assessment Based on Test Result

In some embodiments, some factors may be considered by the AD system when interpreting the result of the PDT test (as described by Sections B and C above), particularly when the subject or patient is a child:
Does the child have the concept of “same” versus “different”? For example, was the child able to indicate when the two teddy bears are of different colors versus when the two teddy bears are of the same color?
Was the child able to point to the “same” and “different” bears without any sound? or was the child only able to show what is same and different?
When xylophone is used to generate audio tones in front of the child, can the child indicate “same” versus “different” with visual and auditory cues? Or can the child indicate “same” versus “different” with just auditory cues?
How easily does the child learn the standardized practice?
What was the score during the PDT test? Were responses automatic and accurate, or no? Did this child use rising inflection as if unsure?

In some embodiments, these factors are observed by the human operating the AD system, interpreted by the operator, and reported to the AD system for the AD system to be analyzed and used to determine treatment for the patient. In some embodiments, some of these factors are observed and interpreted by a computing device implementing the AD system. The computing system implementing AD system may include a machine learning or artificial intelligence system that is trained to interpret interaction with children according to these factors.

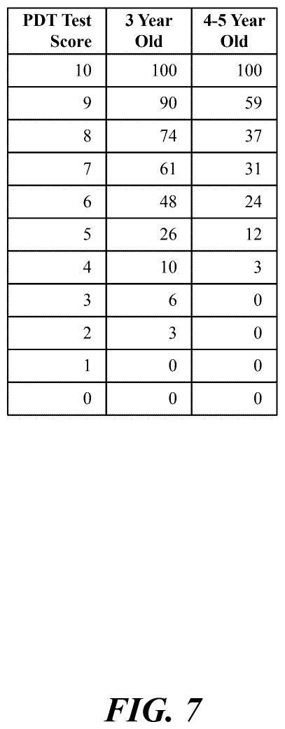

In some embodiments, the raw PDT test score (e.g., as reported in form 420 shown in FIG. 4) is mapped to an age-based percentile. FIG. 7 shows an example aged-based percentile mapping from PDT test scores. The percentile mapping may be a form that is used by a human operator of the AD system (e.g., the therapist). The percentile mapping may also be used by a computing system implementing the AD system to generate a report of the patient based on the patient's raw PDT score.

In some embodiments, the AD system may make treatment recommendations directly based on the raw PDT test score and the age of the patient with or without the percentile mapping. The following is an example guideline for such age-based recommendations:
At age 3, any score is acceptable and so the counseling information regarding what the parents need to teach the child will be less. (The counseling information starts with the concept development of same and different to making judgments about the pitch of two tone stimuli.)
At age 4, scores better than 90% are required to be acceptable as pass, since four-year-olds are starting to be good at this task. Counseling to improve auditory discrimination via auditory training may be recommended if the raw PDT test score is 90% or below. For scores at around 90% (e.g., 9 out 10 trials), the recommendation is to improve automaticity and accuracy for pitch discrimination. For scores at around 80% (e.g., 8 out of 10) or below, the recommendation focuses upon auditory training of pitch discrimination. In some embodiments, supplemental tracks of auditory stimuli are provided by the AD system to parents or professionals to be used for auditory discrimination training of the subject.
At age 5, scores of 100% is expected. Auditory training is recommended if the PDT raw scores is less than 100%. In some embodiments, supplemental tracks of auditory stimuli are provided by the AD system to parents or professionals to be used for auditory discrimination training of the subject.

The recommended auditory training may include home program auditory training. The training may use musical instruments such as a piano or keyboard to generate live stimuli. Smart phone apps that teach pitch discrimination for the purpose of musical training can also be used. A follow-up test with the audiologist after 4 weeks of training may also be recommended.

### E. Treatment Based on PDT

The PDT is part of a comprehensive approach including testing and training materials to evaluate auditory processing skills with stimuli that provide the acoustic requirements for speech perception without the linguistic content. All speech and music are comprised of three acoustic ingredients: loudness, pitch and timing. The unique value of the PDT is providing a test of auditory discrimination for the acoustic requirements of speech perception without the linguistic content. PDT is formed based on the frequencies of speech and the timing demands of speech. The PDT test results play a crucial role in facilitating treatment for auditory processing disorders in several ways:
(1) Diagnosis: The PDT test results provide information about the patient's ability to discriminate if two tones are the same pitch or if they are different pitches. This ability is a foundational skill required for the patient to understand speech. Children develop this skill at an early age. PDT test normative data shows that development of this skill is significant with age and that most typically developing children are likely to develop this skill by the time they are 5 years old. The PDT test results enable audiologists and other members of a multidisciplinary team to make an accurate diagnosis if there is a deficit in pitch discrimination skills.
(2) Treatment planning: The PDT test results help guide audiologists and other members of the multidisciplinary team to determine the most appropriate treatment strategy for each individual patient.
(3) Early detection: The PDT is a simple test that can be used for early detection in developmental age children as young as four to allow for normal child development. There is a critical need for early intervention and therapy for auditory processing disorders. Next to detection, auditory pitch discrimination is the most fundamental auditory process required for developing communication skills (hear it, speak it, read it, write it).
(4) Identification of underlying conditions: Deficits with auditory processing are co-morbid with other learning disabilities such as Dyslexia, ADHD and Autism Spectrum Disorders. Auditory processing disorders are the cause of listening difficulties in many children and can and should be corrected. The PDT provides valuable diagnostic information regarding auditory status and would otherwise be undiscovered. Unless pitch discrimination difficulties are evaluated without linguistic content, it cannot be determined if there is an underlying auditory deficit. The PDT provides such an evaluation of pitch discrimination without using linguistic content.

(5) Results of the PDT provide the basis of a treatment plan for auditory training to correct auditory discrimination difficulties. Musical training, pitch discrimination training and parent education may be components of a treatment plan which can be individualized for each patient, depending on their results, presenting symptoms and behaviors.

(6) Monitoring progress: Follow up testing using the PDT test over time can determine the efficacy during treatment, and make necessary adjustments to the treatment plan, if needed.

(7) Referrals: Based on the results of the PDT test, audiologists can develop treatment plans and use a multidisciplinary approach to remediation, making appropriate referrals when indicated.

(8) Effectuate Medical Treatment: The PDT is normed on children ages three to five and can detect auditory processing difficulties early such that intervention can play a critical role in neural plasticity remediating or minimizing auditory discrimination difficulties.

By providing crucial diagnostic information, auditory processing tests such as the PDT test enable audiologists and the multidisciplinary team members to develop targeted plans for developing auditory discrimination skills, improving overall patient care and outcomes in the management of communication related issues.

### F. Example Electronic System

Many of the above-described features and applications are implemented as software processes that are specified as a set of instructions recorded on a computer readable storage medium (also referred to as computer readable medium). When these instructions are executed by one or more computational or processing unit(s) (e.g., one or more processors, cores of processors, or other processing units), they cause the processing unit(s) to perform the actions indicated in the instructions. Examples of computer readable media include, but are not limited to, CD-ROMs, flash drives, random-access memory (RAM) chips, hard drives, erasable programmable read only memories (EPROMs), electrically erasable programmable read-only memories (EEPROMs), etc. The computer readable media does not include carrier waves and electronic signals passing wirelessly or over wired connections.

In this specification, the term “software” is meant to include firmware residing in read-only memory or applications stored in magnetic storage which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the present disclosure. In some embodiments, the software programs, when installed to operate on one or more electronic systems, define one or more specific machine implementations that execute and perform the operations of the software programs.

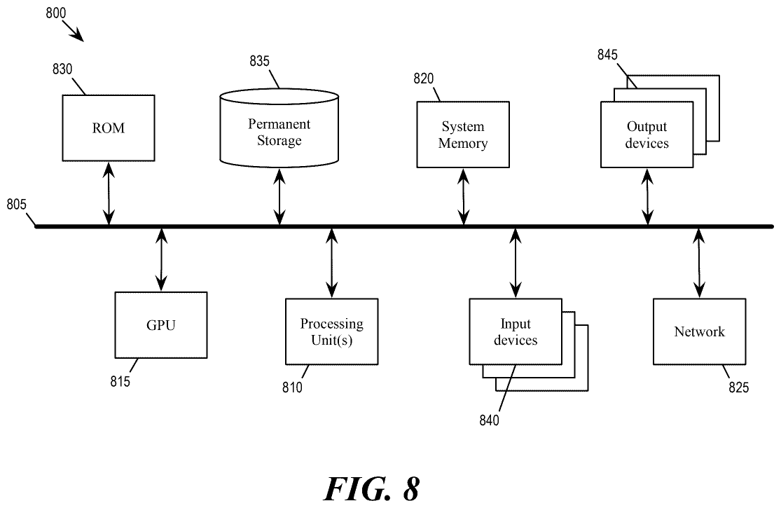

FIG. 8 conceptually illustrates an electronic system 800 with which some embodiments of the present disclosure are implemented. The electronic system 800 may be a computer (e.g., a desktop computer, personal computer, tablet computer, etc.), phone, PDA, or any other sort of electronic device. Such an electronic system includes various types of computer readable media and interfaces for various other types of computer readable media. Electronic system 800 includes a bus 805, processing unit(s) 810, a graphics-processing unit (GPU) 815, a system memory 820, a network 825, a read-only memory 830, a permanent storage device 835, input devices 840, and output devices 845.

The bus 805 collectively represents all system, peripheral, and chipset buses that communicatively connect the numerous internal devices of the electronic system 800. For instance, the bus 805 communicatively connects the processing unit(s) 810 with the GPU 815, the read-only memory 830, the system memory 820, and the permanent storage device 835.

From these various memory units, the processing unit(s) 810 retrieves instructions to execute and data to process to execute the processes of the present disclosure. The processing unit(s) may be a single processor or a multi-core processor in different embodiments. Some instructions are passed to and executed by the GPU 815. The GPU 815 can offload various computations or complement the image processing provided by the processing unit(s) 810.

The read-only-memory (ROM) 830 stores static data and instructions that are used by the processing unit(s) 810 and other modules of the electronic system. The permanent storage device 835, on the other hand, is a read-and-write memory device. This device is a non-volatile memory unit that stores instructions and data even when the electronic system 800 is off. Some embodiments of the present disclosure use a mass-storage device (such as a magnetic or optical disk and its corresponding disk drive) as the permanent storage device 835.

Other embodiments use a removable storage device (such as a floppy disk, flash memory device, etc., and its corresponding disk drive) as the permanent storage device. Like the permanent storage device 835, the system memory 820 is a read-and-write memory device. However, unlike storage device 835, the system memory 820 is a volatile read-and-write memory, such a random access memory. The system memory 820 stores some of the instructions and data that the processor uses at runtime. In some embodiments, processes in accordance with the present disclosure are stored in the system memory 820, the permanent storage device 835, and/or the read-only memory 830. For example, the various memory units include instructions for processing multimedia clips in accordance with some embodiments. From these various memory units, the processing unit(s) 810 retrieves instructions to execute and data to process in order to execute the processes of some embodiments.

The bus 805 also connects to the input and output devices 840 and 845. The input devices 840 enable the user to communicate information and select commands to the electronic system. The input devices 840 include alphanumeric keyboards and pointing devices (also called “cursor control devices”), cameras (e.g., webcams), microphones or similar devices for receiving voice commands, etc. The output devices 845 display images generated by the electronic system or otherwise output data. The output devices 845 include printers and display devices, such as cathode ray tubes (CRT) or liquid crystal displays (LCD), as well as speakers or similar audio output devices. Some embodiments include devices such as a touchscreen that function as both input and output devices.

Finally, as shown in FIG. 8, bus 805 also couples electronic system 800 to a network 825 through a network adapter (not shown). In this manner, the computer can be a part of a network of computers (such as a local area network (“LAN”), a wide area network (“WAN”), or an Intranet, or a network of networks, such as the Internet. Any or all components of electronic system 800 may be used in conjunction with the present disclosure.

Some embodiments include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD−RW, DVD+RW, etc.), flash memory (e.g., SD cards, mini-SD cards, micro-SD cards, etc.), magnetic and/or solid state hard drives, read-only and recordable Blu-Ray® discs, ultra-density optical discs, any other optical or magnetic media, and floppy disks. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, many of the above-described features and applications are performed by one or more integrated circuits, such as application specific integrated circuits (ASICs) or field programmable gate arrays (FPGAs). In some embodiments, such integrated circuits execute instructions that are stored on the circuit itself. In addition, some embodiments execute software stored in programmable logic devices (PLDs), ROM, or RAM devices.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium,” “computer readable media,” and “machine readable medium” are entirely restricted to tangible, physical objects that store information in a form that is readable by a computer. These terms exclude any wireless signals, wired download signals, and any other ephemeral signals.

While the present disclosure has been described with reference to numerous specific details, one of ordinary skill in the art will recognize that the present disclosure can be embodied in other specific forms without departing from the spirit of the present disclosure. In addition, one or more figures (including FIG. 2) conceptually illustrate processes. The specific operations of these

processes may not be performed in the exact order shown and described. The specific operations

may not be performed in one continuous series of operations, and different specific operations may be performed in different embodiments. Furthermore, the process could be implemented using several sub-processes, or as part of a larger macro process. Thus, one of ordinary skill in the art would understand that the present disclosure is not to be limited by the foregoing illustrative details, but rather is to be defined by the appended claims.

The herein-described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Further, with respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Moreover, it will be understood by those skilled in the art that, in general, terms used herein, and especially in the appended claims, e.g., bodies of the appended claims, are generally intended as “open” terms, e.g., the term “including” should be interpreted as “including but not limited to,” the term “having” should be interpreted as “having at least,” the term “includes” should be interpreted as “includes but is not limited to,” etc. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to implementations containing only one such recitation, even when the same claim includes the introductory phrases “one or more” or “at least one” and indefinite articles such as “a” or “an,” e.g., “a” and/or “an” should be interpreted to mean “at least one” or “one or more;” the same holds true for the use of definite articles used to introduce claim recitations. In addition, even if a specific number of an introduced claim recitation is explicitly recited, those skilled in the art will recognize that such recitation should be interpreted to mean at least the recited number, e.g., the bare recitation of “two recitations,” without other modifiers, means at least two recitations, or two or more recitations. Furthermore, in those instances where a convention analogous to “at least one of A, B, and C, etc.” is used, in general such a construction is intended in the sense one having skill in the art would understand the convention, e.g., “a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, e.g., “a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

From the foregoing, it will be appreciated that various implementations of the present disclosure have been described herein for purposes of illustration, and that various modifications may be made without departing from the scope and spirit of the present disclosure. Accordingly, the various implementations disclosed herein are not intended to be limiting, with the true scope and spirit being indicated by the following claims.

What is claimed is:

1. A method comprising:
verifying a subject can indicate when two audio tones are the same in pitch versus when two audio tones are different in pitch;
providing a first set of N trials, each trial comprising two audio tones;
teaching the subject to indicate which of the first set of N trials have two same-frequency audio tones and which of the N trials have two different-frequency audio tones;
providing a second set of M trials, each trial comprising two audio tones, M being at least twice large as N;
receiving an assessment based on the subject correctly indicating which of the second set of M trials have two same-frequency tones and which trial have two different-frequency tones; and
prescribing an audio discrimination training system for the subject based on the assessment.

2. The method of claim 1, further comprising verifying the subject can indicate when two objects are visually the same versus when two objects are visually different before teaching the subject to indicate when two audio tones are the same in pitch versus when two audio tones are different in pitch.

3. The method of claim 2, wherein the two objects are visually presented at a screen of a computing device, and the two audio tones are audibly presented at a speaker of the computing device.

4. The method of claim 2, wherein the two objects are physical items and the two audio tones are generated by physical instruments.

5. The method of claim 1, wherein N≤4 and M≥10.

6. The method of claim 1, wherein the subject is a child in an age range of 3 to 5 years.

7. The method of claim 1, wherein the first and second sets of trials comprise at least one trial having two audio tones that differ in frequency by more than 750 Hertz and less than 1250 Hertz.

8. The method of claim 1, wherein the first and second sets of trials comprise at least one trial having two audio tones that differ in frequency by less than 750 Hertz but more than 250 Hertz.

9. The method of claim 1, wherein first and second sets of trials comprise audio tones in a frequency range between 500 Hertz and 3000 Hertz.

10. The method of claim 1, wherein the assessment comprises a percentile score based on the subject's age.

11. The method of claim 1, wherein treating the subject comprises monitoring the subject by periodically performing a pitch discrimination test with the subject.

12. The method of claim 11, wherein the pitch discrimination test comprises:
providing a plurality of trials, each trial comprising two audio tones; and
receiving an assessment based on the subject correctly indicating which of the plurality of trials have two same-frequency tones and which of the plurality of trials have two different-frequency tones.

13. The method of claim 1, wherein providing the first and second sets of trials comprises playing at least some of the audio tracks on a computing device.

14. The method of claim 1, wherein providing the first and second sets of trials comprises storing at least some of the trials in a server for download into a computing device for playback of the trials.

15. The method of claim 1, wherein the audio tones of the first set of N trials are presented to the subject in conjunction with a visual cue.

16. The method of claim 1, further comprising providing a test to determine how well the subject can indicate when two audio tones are the same in pitch versus when two audio tones are different in pitch.

17. A method comprising:
verifying a subject can indicate when two audio tones are the same in pitch versus when two audio tones are different in pitch;
providing a first set of N trials, each trial comprising two audio tones;
teaching the subject to indicate which of the first set of N trials have two same-frequency audio tones and which of the N trials have two different-frequency audio tones;
providing a second set of M trials, each trial comprising two audio tones, M being at least twice large as N;
receiving an assessment based on the subject correctly indicating which of the second set of M trials have two same-frequency tones and which trial have two different-frequency tones; and
treating a hearing issue of the subject based on the assessment.

18. A method comprising:
verifying a subject can indicate when two audio tones are the same in pitch versus when two audio tones are different in pitch;
providing a first set of N trials, each trial comprising two audio tones;
teaching the subject to indicate which of the first set of N trials have two same-frequency audio tones and which of the N trials have two different-frequency audio tones;
providing a second set of M trials, each trial comprising two audio tones, M being at least twice large as N;
receiving an assessment based on the subject correctly indicating which of the second set of M trials have two same-frequency tones and which trial have two different-frequency tones; and
performing an audio discrimination training session with the subject based on the assessment.
